(19) United States
(12) Patent Application Publication
(10) Pub. No.: US 2009/0053793 A1
(43) Pub. Date: Feb. 26, 2009

(54) Ethanol resistant and furfural resistant strains of E. coli FBR5 for production of ethanol from cellulosic biomass

(75) Inventors: Brian Gerald Lefebvre, Mullica Hill, NJ (US); Mariano Javier Savelski, Sewell, NJ (US); Gregory B. Hecht, Turnersville, NJ (US)

Correspondence Address: JOHN F. LETCHFORD, ARCHER & GREINER, P.C., ONE CENENNIAL SQUARE, HADDONFIELD, NJ, 08033, US

(73) Assignee: Rowan University, Glassboro, NJ (US)

(21) Appl. No.: 12/288,414

(22) Filed: Oct. 20, 2008

Related U.S. Application Data
(62) Division of application No. 11/974,447, filed on Oct. 12, 2007.
(60) Provisional application No. 60/851,690, filed on Oct. 13, 2006.
(60) Provisional application No. 60/865,913, filed on Nov. 15, 2006.

(51) Int. Cl. C12N 1/20

(52) U.S. Cl. 435/252.8

(57) ABSTRACT

Ethanol and furfural challenged strains of E. coli FBR⁵ exhibiting higher ethanol yield, productivity, and tolerance to both ethanol and furfural than FBR⁵ and methods for producing same.

### CROSS REFERENCE TO RELATED APPLICATIONS

This application is a divisional of U.S. patent application Ser. No. 11/974,447, filed Oct. 12, 2007 which claims the benefit of U.S. Provisional Patent Application No. 60/851,690, filed Oct. 13, 2006, and U.S. Provisional Patent Application No. 60/865,913, filed Nov. 15, 2006, both of which are incorporated herein by reference in their entireties.

### FIELD OF THE INVENTION

The present invention relates in general to deriving fuel-grade ethanol from cellulosic biomass and, in particular, to deriving fuel-grade ethanol from cellulosic biomass using ethanol resistant and furfural resistant strains of E. coli FBR5.

### BACKGROUND OF THE INVENTION

Ethanol is an environmentally friendly alternative to conventional fossil fuel derivatives such as gasoline. That is, it produces less harmful exhaust products upon combustion than gasoline or similar combustion engine fuels. Consequently, ethanol is becoming an increasingly accepted supplement to gasoline for use in internal combustion engine vehicles, i.e., 10-15% ethanol/85-90% gasoline formulations are becoming a more common source of fuel for such vehicles. While ethanol may be obtained from fossil fuels, which are a finite natural resource, it is increasingly being produced from renewable sources such as corn grain. The conversion of corn grain to ethanol is an established practice. However, the capital investment for producing ethanol is currently about $1.00-$1.50 per gallon of annual capacity in the United States (“US”). Presently, the biggest hindrance to increasing production of ethanol is the ability to use a biomass feedstock for fermentation that is plentiful and inexpensive. Residual agricultural biomass represents an largely untapped resource for renewable fuel production. The most abundant and inexpensive biomass feedstocks are those in which the sugars are derived of lignocellulose, such as corn stover, wood chips, grasses, straws and other agricultural residues. Among these, corn stover represents the largest quantity of agricultural residue available in the US. Every year a dry weight of 250 million tons of corn stover is available, with between 50%-66% of that being available for use in ethanol fermentation. As used herein, the term “corn stover” means the residue that remains after harvesting of corn grain, i.e., the dried stems, stalks and leaves of the corn plant. In 2005, the US produced nearly 4 billion gallons of ethanol for fuel. At the time of this writing, the current capacity of ethanol production in the US is nearly 4.5 billion gallons per year with another potential of nearly 2 billion gallons per year becoming available in the near future via ethanol production facilities under construction. At approximately 68 gallons of ethanol produced per ton of corn stover, collection and conversion of half of all available corn stover could result in 8.5 billion gallons of ethanol production, nearly triple the current production of ethanol from corn grain. For corn stover prices of $25 per dry ton delivered and 3 tons of corn stover removed for each acre of corn grown, farmer net income could increase by $20/acre of corn in present dollars.

Heretofore, ethanol production from lignocellulosic biomass was not economically feasible, in part due to limitations in biocatalyst performance. Obtaining a high ethanol yield from lignocellulosic biomass requires the use of a biocatalyst that rapidly produces ethanol with few byproducts, metabolizes all sugars produced by biomass treatment and resists toxins present in the feedstock and the fermentor. Lignocellulosic hydrolysates contain a mixture of sugars, including both hexose and pentose sugars. Hexose sugars are sugars with six carbon molecules, such as glucose, while pentose sugars are sugars with five carbon molecules, such as xylose. Traditional microorganisms used for ethanol fermentation, such as Saccharmyces cerevisiae and Zymomonas mobilis, do not metabolize pentoses. Some microorganisms, like Escherichia coli (E. coli) and Klebsiella oxytoca, are naturally able to ferment a wide range of sugars. Ideal characteristic requirements for an industrially suitable microorganism for ethanol production can be seen in Table 1.

TABLE 1
Important Traits for Ethanol Production

| Trait | Requirement |
|---|---|
| Ethanol Yield | >90% of theoretical |
| Ethanol Tolerance | >40 g/L |
| Ethanol Productivity | >1 g/Lh |
| Robust Grower and Simple | Inexpensive Medium |
| Growth Requirements | Formulation |
| Able to Grow in | Resistance to Inhibitors |
| Undiluted Hydrolysates | |
| Culture Growth Conditions | Acidic pH or Higher |
| Retard Contaminants | Temperatures |

E. coli has several advantages as a biocatalyst for ethanol production. Not only does it have the ability to ferment many different types of sugars, it also has no requirements for complex growth factors and it has prior industrial use for the production of recombinant protein. The major disadvantages of E. coli are a narrow and neutral pH growth range, less hardy cultures compared to yeast, biotoxicity, and negative public perceptions regarding the danger of E. coli strains. Over the past two decades, extensive research and development of E. coli has produced derivative strains that selectively produce ethanol from both pentose and hexose sugars. One strain in particular, FBR5, has many desirable characteristics, one of which is its efficiency at producing ethanol. FBR5 has been engineered for ethanologenic fermentation, can ferment and grow on both pentose and hexose sugars, and is genetically stable, unlike some other ethanologenic E. coli strains. The presently understood characteristics of FBR5 for ethanol fermentation are seen in Table 2.

TABLE 2
E. coli FBR5 Fermentation Characteristics

| Trait | Literature Value |
|---|---|
| Ethanol Yield | 90% |
| Maximum Ethanol | 41.5 g/L |
| Ethanol Productivity | 0.59 g/Lh |

The desired byproduct of fermentation, ethanol, is very toxic and, once enough has accumulated in the fermentor, cell growth slows and detrimentally affects the overall ethanol yield. Thus, the performance of presently available ethanol tolerant biocatalysts are adversely affected by the ethanol they produce.

In addition, when using corn stover as a feedstock, a complex dilute-acid pretreatment is a common method used to release usable sugars from the hemicellulose. During this pretreatment process toxic byproducts are produced. One major inhibitory toxin produced during this process is the aldehyde furfural. Furfural is very similar in structure to xylose and very difficult to separate from the hydrolysate. Furfural is detrimental to the growth of cells and therefore negatively-impacts the production of ethanol.

An advantage exists, therefore, for a system and method for producing high yield ethanol from an abundant lignocellulosic biomass using an ethanol resistant biocatalyst.

A further advantage exists for a system and method for producing high yield ethanol from an abundant lignocellulosic biomass using a highly ethanol resistant strain of E. coli.

A further advantage exists for a system and method for producing high yield ethanol from an abundant lignocellulosic biomass using a highly furfural resistant E. coli.

A further advantage exists for a system and method for producing high yield ethanol from an abundant lignocellulosic biomass using a highly ethanol and furfural resistant E. coli.

### SUMMARY OF THE INVENTION

According to the invention, ethanol resistant derivatives of E. coli FBR5 have been grown and isolated and identification of mutant strain characteristics have been studied through pilot fermentation experiments. Using the systems and methods of the invention, derivative E. coli FBR5 strains have been developed with higher ethanol yield, productivity, and tolerance to both ethanol and furfural.

For example, during pilot fermentor studies in a Luria-Bertani (LB) broth medium containing 150 g/L xylose, derived strains of E. coli identified herein as “ARL” and “ANE” produced over 50 g/L of ethanol while “parent” E. coli FBR5 produces roughly 40 g/L of ethanol. Further fermentations were performed with the goal of maximizing ethanol concentration. However, it was observed that very high concentrations of xylose (>175 g/L) were found to inhibit cell growth and ethanol production. The fed-batch strategy combines the high ethanol yields and rapid ethanol production observed in batch fermentations with a product stream at a high ethanol concentration.

The methods according to the invention have also produced strains of E. coli FBR5 that demonstrate increased furfural resistance relative to FBR5 as well as strains that possess dual resistance to both ethanol and furfural. Consequently, the novel E. coli FBR5 strains according to the invention overcome considerable obstacles which heretofore have hindered economical use of lignocellulosic biomass, particularly corn stover, as a viable bioethanol source material.

Other details, objects and advantages of the present invention will become apparent as the following description of the presently preferred embodiments and presently preferred methods of practicing the invention proceeds.

### BRIEF DESCRIPTION OF THE DRAWINGS

The invention will become more readily apparent from the following description of preferred embodiments thereof shown, by way of example only, in the accompanying drawings wherein:

FIG. 1 is a schematic representation of a typical bioethanol production process;

FIGS. 2-8 represent a sequence by which ethanol resistant and furfural resistant strains of E. coli FBR5 are derived in accordance with the present invention;

FIG. 9 is a graph of ethanol concentration, xylose concentration, and cell optical density over time for E. coli FBR5 under batch fermentation at 100 g/L xylose;

FIG. 10 is a graph of ethanol concentration, xylose concentration, and cell optical density over time for E. coli FBR5, E. coli ARL, and E. coli ANE under batch fermentation at 150 g/L xylose;

FIG. 11 is a graph of ethanol concentration for E. coli ARL under fed batch fermentation maintained at 100 g/L xylose;

FIG. 12 is a graph of ethanol concentration, xylose concentration and glucose concentration for E. coli ARL and E. coli FBR5 under fed batch fermentation at 57 g/L xylose and 43 g/L glucose;

FIG. 13 is a xylose calibration curve in a 50 μL sample xylose in water plotted at a light absorbance frequency of 554 nm;

FIG. 14 is a graph depicting ethanol yield and relative changes in luminescence over time for E. coli FBR5 at 30° C.;

FIG. 15 is similar to FIG. 14 depicting ethanol yield and relative changes in luminescence over time for E. coli ANA at 30° C.;

FIG. 16 is similar to FIG. 14 depicting ethanol yield and relative changes in luminescence over time for E. coli ARP at 30° C.;

FIG. 17 is similar to FIG. 14 depicting ethanol yield and relative changes in luminescence over time for E. coli ARL at 30° C.;

FIG. 18 is similar to FIG. 14 depicting ethanol yield and relative changes in luminescence over time for E. coli ANE at 30° C.;

FIG. 19 is similar to FIG. 14 depicting ethanol yield and relative changes in luminescence over time for E. coli FBR5 at 35° C.;

FIG. 20 is similar to FIG. 14 depicting ethanol yield and relative changes in luminescence over time for E. coli ARL at 35° C.;

FIG. 21 is similar to FIG. 14 depicting ethanol yield and relative changes in luminescence over time for E. coli ANE at 35° C.;

FIG. 22 is a graph of the optical density, ethanol production and xylose concentration under fermentation with E. coli ARL over time in the presence of 150 g/L xylose at 35° C.;

FIG. 23 is a graph of the optical density, ethanol production and xylose concentration under fermentation with E. coli ANE over time in the presence of 150 g/L xylose at 35° C.;

FIG. 24 is a graph of the optical density, ethanol production and xylose concentration under fermentation with E. coli FBR5 over time in the presence of 150 g/L xylose at 35° C.;

FIG. 25 is a table summarizing the ethanol performance characteristics of ideal strains of microorganisms and actual strains of microorganisms studied pursuant to the present invention;

FIG. 26 is a graph of the optical density and ethanol production under fermentation with E. coli FBR5 over time in the presence of 100 g/L xylose and 1.5 g/L furfural at 35° C.;

FIG. 27 is a graph of the optical density and ethanol production under fermentation with E. coli PS6 over time in the presence of 100 g/L xylose and 1.5 g/L furfural at 35° C.;

FIG. 28 is a table summarizing the ethanol performance characteristics of E. coli FBR5 and E. coli PS6 in the presence of 100 g/L xylose and 1.5 g/L furfural at 35° C.;

FIG. 29 is a graph of the relative growth of E. coli PS6 versus E. coli FBR5 as a function of exposure to ethanol;

FIG. 30 is a graph of ethanol production and xylose concentration under fermentation of E. coli PS6 versus E. coli FBR5 over time in the presence of approximately 100 g/L xylose and 1.5 g/L furfural;

FIG. 31 is a graph of ethanol production and xylose concentration under fermentation of E. coli PM9 versus E. coli FBR5 over time in the presence of approximately 100 g/L xylose and 1.5 g/L furfural; and

FIG. 32 is a graph of the relative growth of E. coli PS6 and PM9 versus E. coli FBR5 as a function of exposure to ethanol.

### DETAILED DESCRIPTION OF THE INVENTION

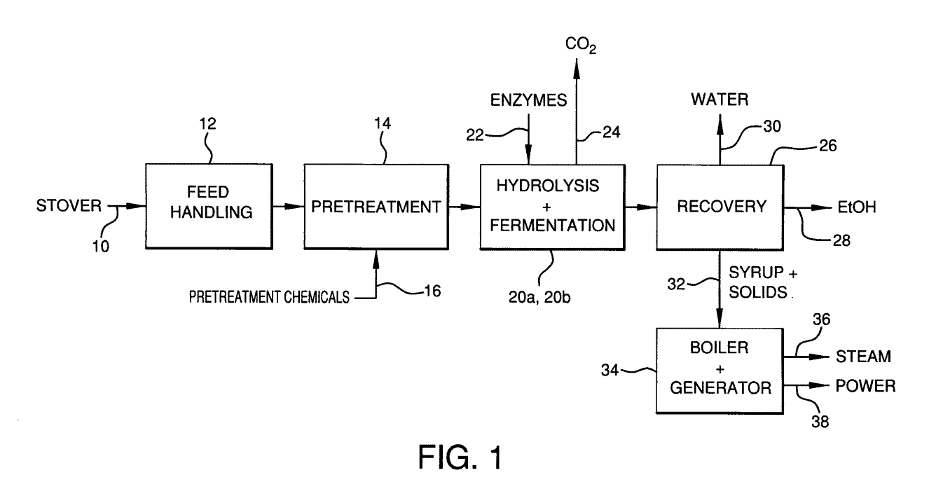

Referring to FIG. 1, there is shown is a schematic representation of a typical bioethanol production process. During feed handling, bales or other quantities of lignocellulosic biomass 10 such as, by way of example but not limitation, corn stover, are initially feed handled at step 12. In typical feed handling the biomass is unwrapped, washed, and milled in preparation for chemical pretreatment at step 14. Biomass must be pretreated to realize high sugar yields that are vital to the commercial success of the process. At minimum, pretreatment prepares cellulose for enzymatic hydrolysis with high yields. Some pretreatments are also effective at releasing monomer sugars from hemicellulose. Any suitable pretreatment chemicals 16 may used be in the pretreatment phase. Typical pretreatment chemicals may include, for example, and without limitation, water, steam, ammonia, one or more acids, including but not limited to sulfuric acid, or other constituents depending on the source biomass and desired pretreatment method. Thereafter, enzymatic hydrolysis of pretreated stover occurs at step 20a to produce glucose from cellulose. Typical enzymes 22 used in enzymatic hydrolysis may include, for example, and without limitation, cellulases, xylanases and amylases. These enzymes produce sugar monomers (glucose, xylose, etc.) from the sugar polymers present in the biomass. The enzymes do not generally work unless the chemical pretreatment step first “opens up” the biomass to enzymatic attack. This completes the work preparation portion of the process.

Ethanol production begins in the fermentation stage 20b. Although they may be performed separately, to minimize capital costs, enzyme hydrolysis 20a and fermentation 20b preferably occur in the same vessel if the enzymes and microorganisms can thrive under common conditions. As indicated by reference numeral 24 carbon dioxide (CO₂) is produced as a by-product of the fermentation process. According to the present invention, the E. coli strains developed herein perform fermentation, i.e., converting simple sugars into ethanol. What the present inventors have discovered is that the E. coli strains of the invention are unique in that they perform well at high sugar concentrations and in the presence of toxic byproducts formed during the most popular chemical pretreatments. The separation and recovery phase begins at step 26 after all sugars are converted to ethanol, thereby generating a pure ethanol (EtOH) stream 28, a water stream 30, and a residual stream 32 of syrup and solids that can be burned at a boiler and generator 34 to generate steam 36 or other motive power 38.

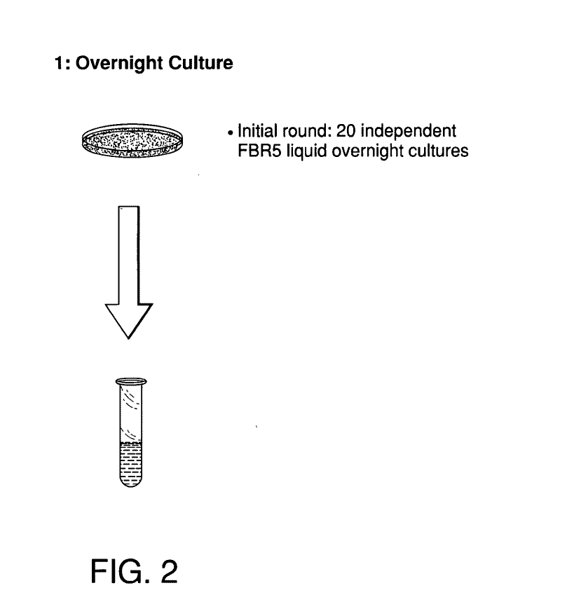

Referring to FIGS. 2-8, there is shown a sequence of steps by which ethanol resistant and furfural resistant strains of E. coli FBR5 are grown in accordance with the present invention. The microorganisms were grown on plates of Luria-Bertani (LB) broth supplemented with xylose and ampicillin. The LB broth used in the studies comprised 10 g of tryptone, 5 g of yeast extract, 10 g of sodium chloride, 15 g of granulated agar per liter and is marketed by Fisher Scientific of Waltham, Mass. It will be understood, however, that any suitable bacteria or culture growth broth or media known to those of ordinary skill in the present art other than LB may be used if desired.

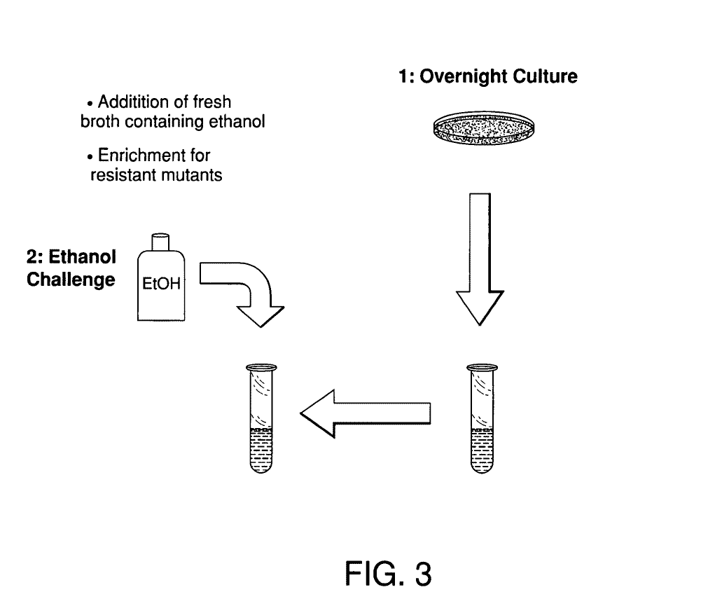

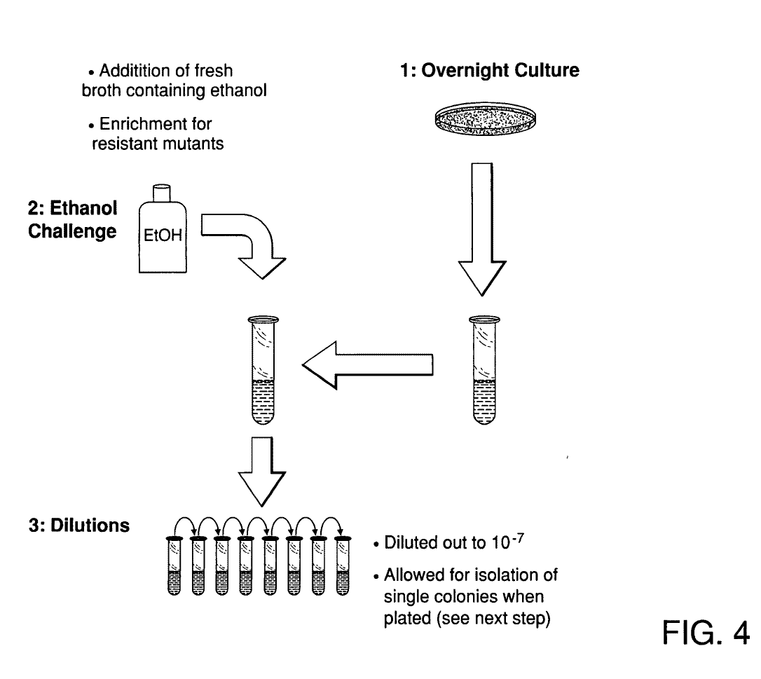

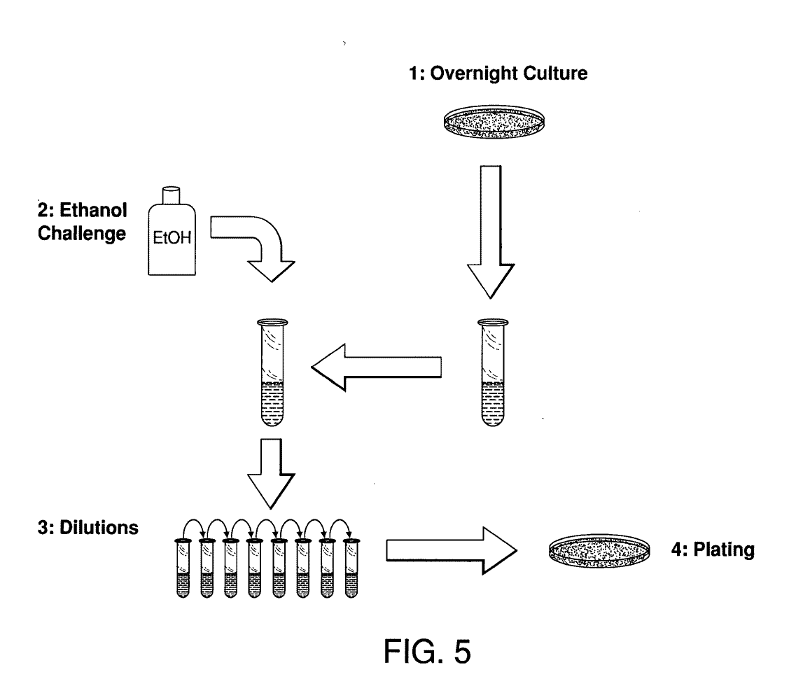

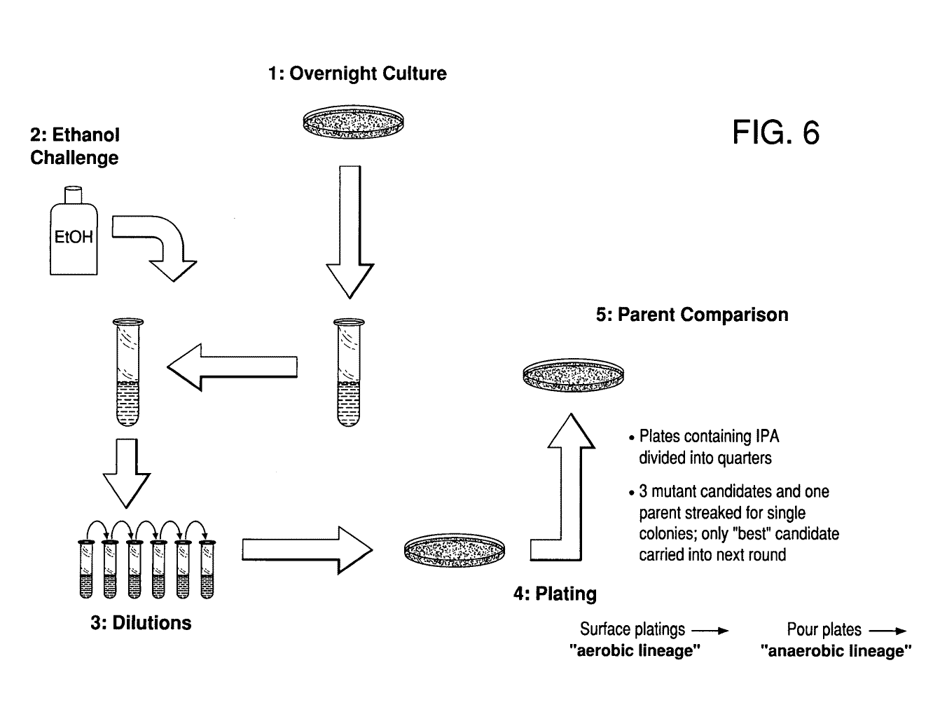

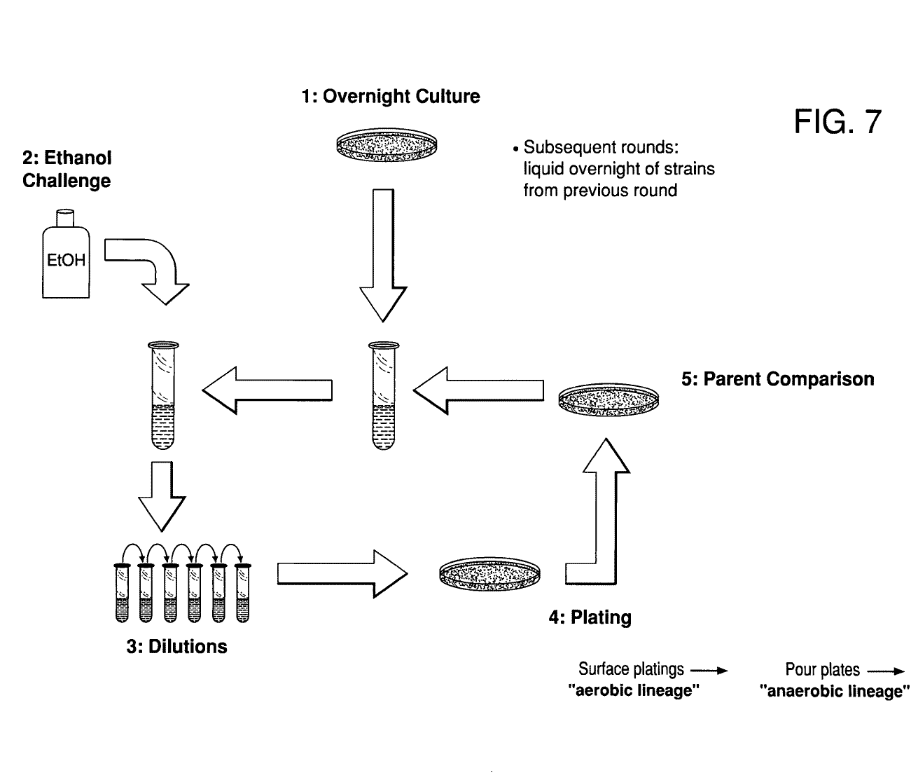

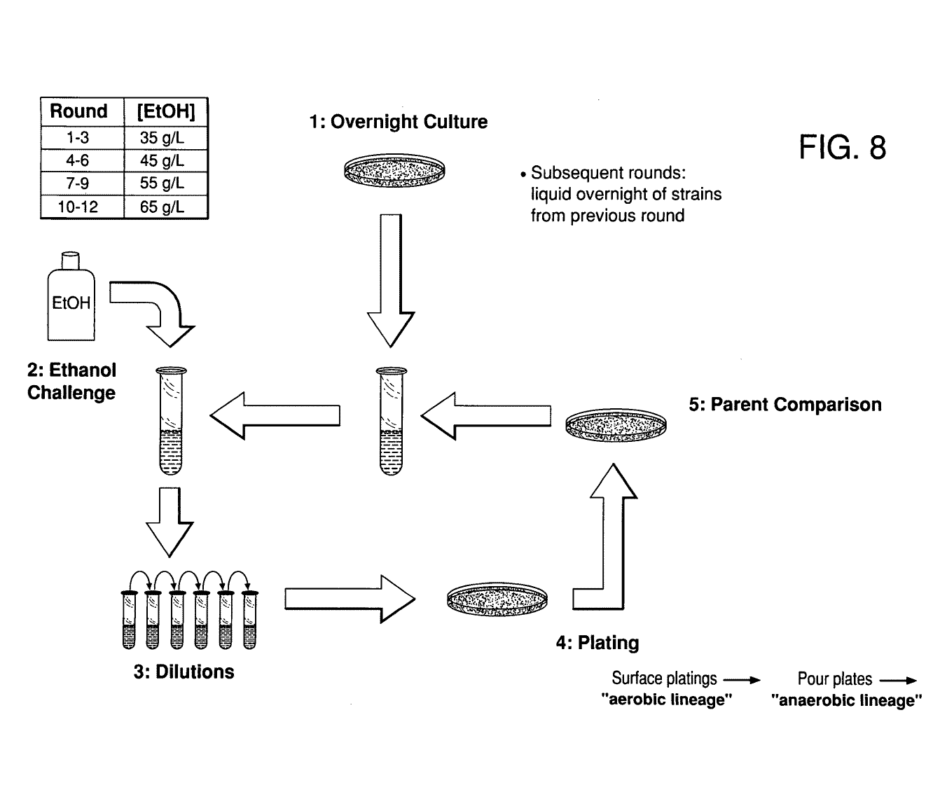

As seen in FIG. 2, an initial round of twenty cultures of a parent strain of E. coli FBR5 were grown in 5 mL of LB containing 20 g/L xylose and 100 mg/ml ampicillin for 24 hours at 30° C. Referring to FIG. 3, after 24 hours the cultures were exposed to “ethanol challenges” wherein fresh ethanol-containing LB broth was added to the cultures (the amounts of ethanol added to this round of cultures and subsequent rounds of derivatives thereof are shown in FIG. 8) along with isopropanol, ampicillin, and xylose that are believed to serve as enrichment for potentially ethanol resistant mutant strains. FIG. 4 shows that the ethanol challenges produced diluted amounts of bacteria to approximately 10⁻⁷ of their original amounts by addition of fresh broth and ethanol which allowed for isolation of single colonies of ethanol resistant strains of FBR5 when plated. As seen in FIG. 5, ten cultures from the serial dilutions were plated on overnight growth plates to simulate an aerobic environment (“aerobic lineage”) while ten others were grown on pour plates which were used to simulate an anaerobic environment (“anaerobic lineage”). The overnight growth plates consisted of the aforementioned LB broth as well as agar in the amount of 1.65%, 20 g/l xylose, 10 g/l isopropanol and 100 mg/L ampicillin. The pour plates consisted of the aforementioned LB broth as well as agar in the amount of 0.35%, 20 g/l xylose, 10 g/l isopropanol and 100 mg/L ampicillin. The two sets of ten cultures were incubated at 30° C. until growth was observed. Turning to FIG. 6, the three largest (mutant) colonies from each lineage were selected for enrichment and were plated against the original parent strain for each of the 20 cultures. The largest growing colonies from the mutant-parent comparison were selected from each lineage for a second round of ethanol resistance development at 35 g/l ethanol and plated on the above-described overnight growth plates. At the same time, the original parent strain (FBR5) was transferred from stock plates containing the above-described LB broth and 1.65% agar, 20 g/l and 100 mg/L ampicillin for purposes of comparison. As seen in FIG. 7, the three largest colonies from the second round of ethanol resistance development were selected for enrichment and were again plated against the original parent strain for each culture. The largest growing colonies from the second round were selected from each lineage for a third round of ethanol resistance at 35 g/l ethanol. The fourth through sixth rounds, seventh through ninth, and tenth through twelfth rounds of ethanol resistance development follow the same procedure as the first through third rounds, except that the ethanol concentration in the media was increased by 10 g/L until it reached 65 g/L, as shown in FIG. 8.

Thereafter, ten mutant strains from the surface media plating and ten mutant strains from the pour plating were isolated from the 65 g/L ethanol rounds and frozen. The nomenclature for the mutant strains depended on how they were derived. More specifically, mutant strains derived from pour plating were labeled AN—for anaerobic derivation followed by a letter designating the order of which they were found up to the letter J. Mutant strains derived from surface media plating were labeled AR—for aerobic derivation and followed by a letter designating the order of which they were found starting at the letter K. Strains “ARL” and “ANE” were studied in the pilot studies.

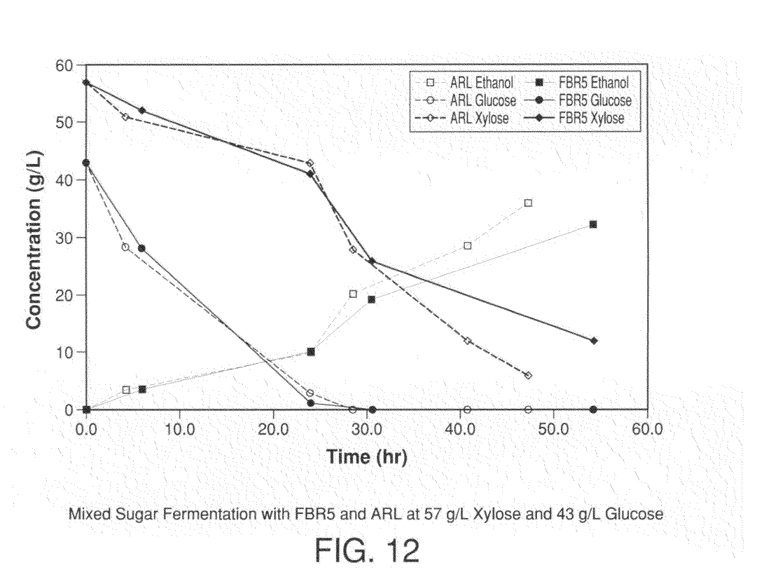

Pilot fermentation studies were conducted in a 3L fermentation vessel in conjunction with a BioFlo 3000 system (which is marketed by New Brunswick Scientific Co. of Edison, N.J.) to monitor temperature, dissolved oxygen, air flow rate, agitation rate and pH. The media used for fermentation consisted of LB broth, xylose at various concentrations, and ampicillin. For some runs, glucose was also added to the media (see the mixed sugar fermentation discussed in connection with FIG. 12 below). To ensure sterility, the fermentor, with the media inside, along with the condenser, and a flask of distilled water were autoclaved the day before each fermentation run. An overnight culture of the strain to be used in the fermentation was then grown the night before each run. The fermentor was connected to the BioFlo 3000 system when ready to begin the run. To connect the fermentor to the BioFlo 3000, the dissolved oxygen probe, the motor, the jacket water in and out passages, and the air flow from the BioFlo 3000 were attached to the fermentor. Thereafter, the condenser was connected to the fermentor, the water flow in and out passages were attached to the condenser, and a tube was connected from the end of the condenser to the flask of distilled water. That ensured that nitrogen was flowing through the fermentor by monitoring bubbling of the water. As long as the water is bubbling, nitrogen is flowing through the system and any oxygen is being purged out. A thermometer was then placed into the thermometer well of the fermentor. Once the fermentor was connected to the BioFlo 3000, the BioFlo 3000 was turned on. The agitation rate was then set up to about 400 rpm and the temperature was set to at least 30° C. or, more preferably, greater, such as, for example, at least about 35° C. The nitrogen and water flows were then opened, whereby the fermentor was ready for the addition of the overnight culture. The following was then added to the fermentor: (1) 300 mL of sterile, filtered LB broth, (2) an amount of xylose determined as a function of the desired initial sugar concentration, (3) an amount of ampicillin such that the initial concentration in the fermentor was always 100 mg/L, and (4) the overnight culture, whereby fermentation began.

An initial sample was collected and analyzed at the start of fermentation, with additional samples collected once every four to six hours (except overnight) until completion of a fermentation run. All samples were analyzed using a YSI biochemistry analyzer (marketed by YSI Inc. of Yellow Springs, Ohio), a spectrophotometer, and a xylose assay.

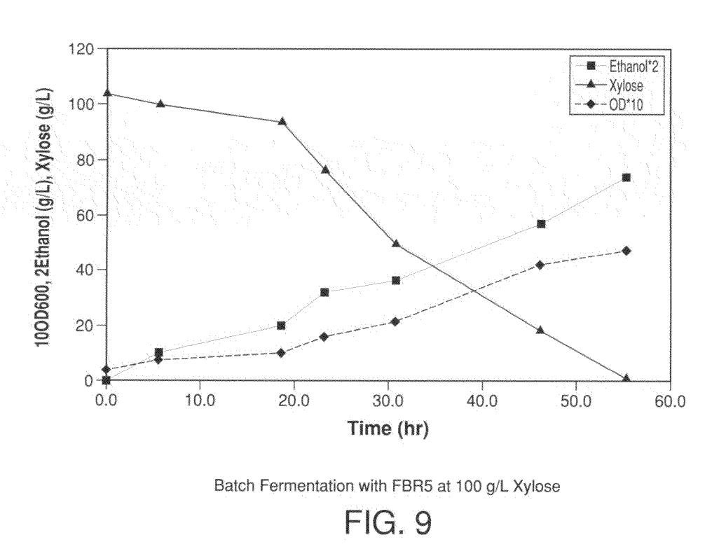

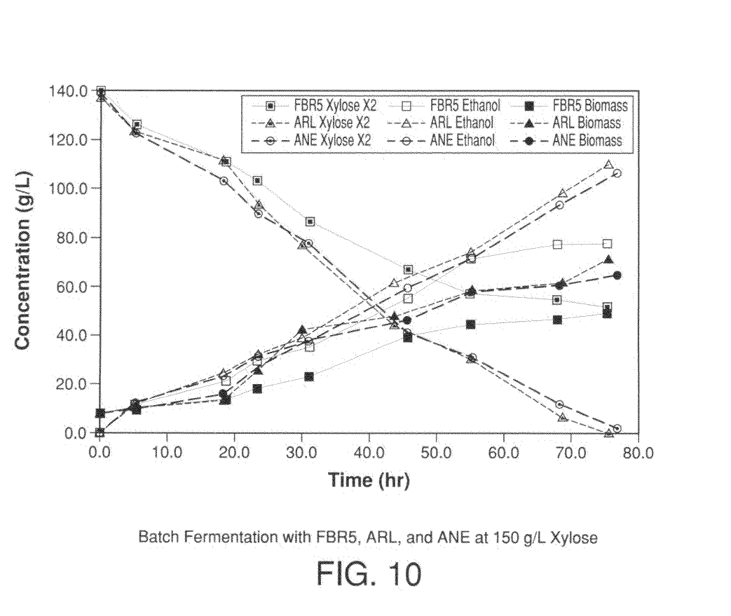

The YSI biochemistry analyzer determines the concentration of glucose and ethanol in a sample. The spectrophotometer is used to determine the cell concentration of the sample by assessing optical density at an absorbance 600 nm over time. Calibration curves were created to determine the cell concentration of the sample based on optical density. The calibration curves were created by sampling a large volume of spent media extracted from the fermentor and placed into a weighed tube. These samples are then spun down using a centrifuge at between about 300-400 rpm. After removing the liquid from the spun down tube and drying the tube, the dry tube with the dense cells at the bottom of the tube was then weighed. The weight of the tube alone was subtracted from that total to give the weight of the cells that was collected in a sample. Once the weight of the cells was known, it was divided by the volume of the sample to give the cell concentration for the given sample. Each sample's cell concentration was then plotted versus its optical density to produce the calibration curve. Optical densities for various FBR strains with various concentrations of sugars (xylose or (xylose and glucose)) are depicted in FIGS. 9-12. In FIG. 9, the optical density (“OD”) is multiplied by a factor of 10 and the ethanol concentration is multiplied by a factor of 2 in order for all of the graphical data to fit on the “y-axis” of the graph. Similarly, in FIG. 10 the concentrations of FBR5 xylose, ARL xylose, ANE xylose, FBR5 biomass, ARL biomass and ANE biomass are multiplied by a factor of 2 in order for all of the graphical data to fit on the “y-axis” of the graph.

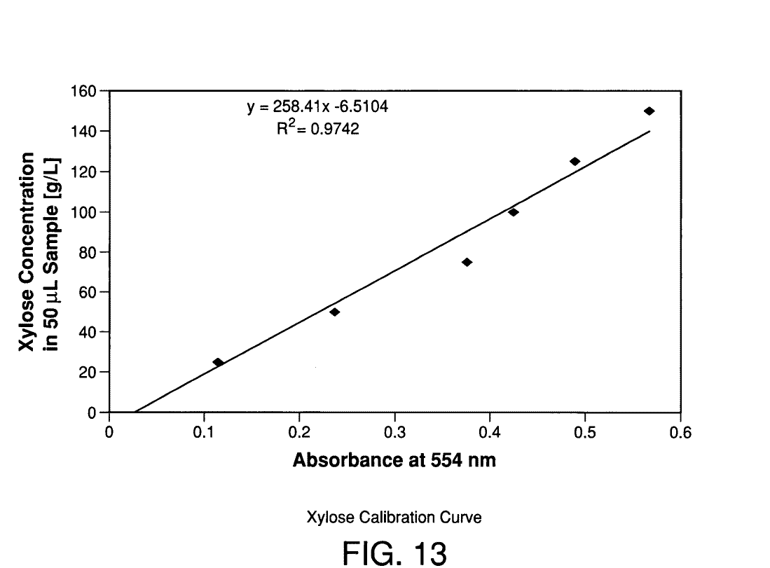

The xylose assay was used to determine the xylose concentration in the sample. To set up the xylose assay, 950 μL of 6 M HCl, 47.5 μL of 0.2% benzoic acid, 2.5 μL of the sample and 5 mL of a color reagent solution were combined in a test tube and mixed vigorously. The color reagent solution consisted of 0.5 g phloroglucinol and 100 mL acetic acid which reacts with aldehyde groups to form a purple color in solution. The sample solution was heated for 5 minutes in boiling water and then cooled in an ice water bath for 5 minutes. Once the sample was cool, a reading of the optical density at absorbance of 554 nm on the spectrophotometer was observed. After the readings were complete, the optical density was used in Equation 1 to determine the actual xylose concentration according to Equation (1):

$$[ X ] = \frac{( OD - 0.0327 )}{0.0038} \qquad Equation ( 1 )$$

where [X] is the xylose concentration in g/L and OD is the optical density at an absorbance of 554 nm. In order to ensure that the equation is correct, a calibration curve should be completed prior to each xylose analysis. An example of a typical xylose calibration curve is shown in FIG. 13.

The first fermentation run according to the invention was conducted at a xylose concentration of 100 g/L with FBR5. This was done to compare the literature values for maximum ethanol concentration, ethanol productivity, and ethanol yield of E. coli FBR5 to the values obtained by the strand of FBR5 that was used in the present invention in isolation of all its mutant derivatives.

Ethanol yield (for this and later references herein) is expressed in Equation (2):

$$Y = \frac{Ethanol produced ( grams )}{\begin{matrix}Sugar consumed , either xylose \\ or ( xylose and glucose ) ( grams )\end{matrix}} = {Y}_{EtOH / S} ( g \text{/} g ) \qquad Equation ( 2 )$$

As seen from Table 3, the values obtained from FBR5 and its literature value (Table 2, supra) are very similar. A graph of the ethanol concentration, xylose concentration, and cell optical density over time for FBR5 under batch fermentation at 100 g/L xylose can be seen in FIG. 9.

TABLE 3
Comparison of Literature and Actual Values of FBR5

Strain

| | FBR5 | FBR5 |
|---|---|---|
| | (Literature) | (Measured) |
| Xylose (g/L) | 95 | 100 |
| Maximum Ethanol (g/L) | 41.5 | 36.9 |
| Ethanol Production (g/Lh) | 0.59 | 0.67 |
| Y_{EtOH/S} (g/g) | 0.44 | 0.36 |

Since the comparison run resulted in similar values between the literature values and the actual values for FBR5, it was decided to conduct runs that compare FBR5 to certain mutant strains thereof. ARL and ANE were chosen for comparison to FBR5 based on prior growth curves conducted for all mutant strands. The xylose concentration for these runs was raised to 150 g/L to ascertain the maximum ethanol concentration that could be achieved by the parent and mutant strains. As seen from Table 4, the values of the maximum ethanol concentration and the ethanol productivity for the mutant strains ARL and ANE were significantly higher than the parent strain FBR5, thereby demonstrating that the mutant strains are more effective at ethanol production. Similarly, a graph of the ethanol concentration, xylose concentration, and cell optical density over time for FBR5, ARL, and ANE under batch fermentation at 150 g/L xylose can be seen in FIG. 10.

TABLE 4
Comparison of Parent Strain to
Mutant Strains at 150 g/L Xylose

| | Strain | | |
|---|---|---|---|
| | FBR5 | ARL | ANE |
| Xylose (g/L) | 150 | 150 | 150 |
| Maximum Ethanol (g/L) | 38.9 | 55.1 | 53.2 |
| Ethanol Production (g/Lh) | 0.52 | 0.73 | 0.69 |
| Y_{EtOH/S} (g/g) | 0.26 | 0.39 | 0.39 |

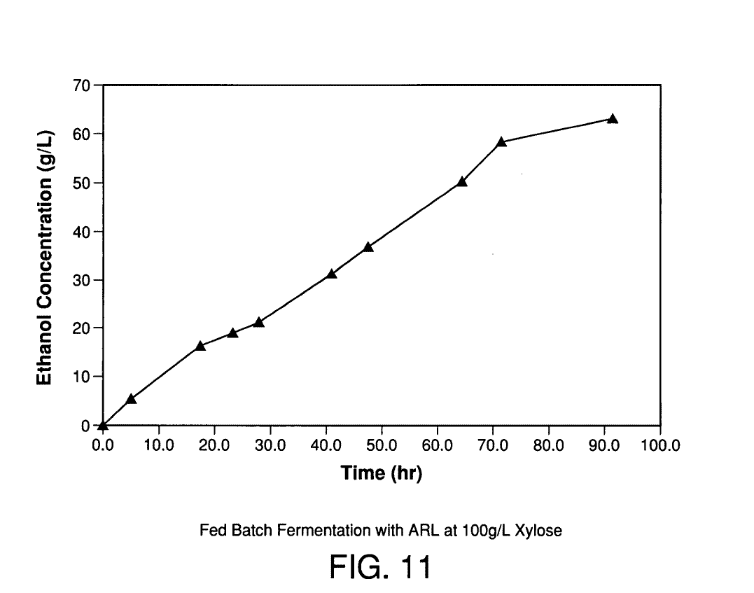

During the 150 g/L xylose runs, it was observed that the ARL and ANE strains consumed all of the xylose but the FBR5 strain did not. This implied that ARL and ANE strains did not reach the maximum ethanol concentration they could produce. To confirm this theory, further fermentation runs were conducted in order to determine the maximum ethanol concentration for those strains. The first fermentation run conducted to determine the maximum ethanol concentration was with ARL at a xylose concentration of 175 g/L. During that run, it was observed that all the xylose was consumed and the maximum ethanol concentration was still not reached. The next fermentation run conducted to determine the maximum ethanol concentration was with ARL at a xylose concentration of 250 g/L. During that run, it was observed that the cells did not grow as well as normally expected, and that this inhibition of growth significantly affected its ethanol production. It was concluded that too high a xylose concentration hinders cell growth, and ultimately, ethanol production. Accordingly, in order to determine essentially maximum ethanol production, fed-batch fermentation at a lower, but maintained concentration of xylose was conducted. Fed-batch fermentation runs were conducted with ARL by maintaining the xylose concentration at 100 g/L daily. Every morning, a sample of the media was collected and analyzed for xylose concentration. Once the current xylose concentration was known within the fermentor, additional xylose in 300 mL of media was added to the fermentor to raise the xylose concentration back up to 100 g/L. Table 5 shows the values obtained for ARL using fed batch fermentation. A graph of ethanol concentration for ARL at a xylose concentration maintained at 100 g/L xylose under fed batch fermentation can be seen in FIG. 11.

TABLE 5
Fed Batch Fermentation with ARL.
Xylose concentration maintained at 100 g/L

| | Strain |
|---|---|
| | ARL |
| Total Xylose (g) | 221 |
| Maximum Ethanol (g/L) | 63.1 |
| Ethanol Production (g/Lh) | 0.69 |
| Y_{EtOH/S} (g/g) | 0.29 |

Mixed sugar fermentation runs were conducted to simulate conditions which are more likely be present in industrial fermentation runs using pretreated corn stover hydrolysates and not synthetic hydrolysates. These runs contained xylose and glucose. The xylose and glucose concentrations at the start of the fermentation were 57 g/L and 43 g/L, respectively. The strains used for the mixed sugar fermentation runs were FBR5 and ARL. As can be seen in Table 6, ARL reach a higher maximum ethanol concentration and ethanol productivity than FBR5. This implied that the mutant strain, ARL, is more effective at ethanol production than the FBR5 parent strain under more realistic “industrial” conditions. A graph of the ethanol concentration, xylose concentration, and glucose concentration over time for each strain can be seen in FIG. 12.

TABLE 6
Mixed Sugar Fermentation with FBR5 and ARL.
Initial xylose and glucose concentrations
were 57 g/L and 43 g/L, respectively.

Strain

| | FBR5 | ARL |
|---|---|---|
| Xylose (g/L) | 57 | 57 |
| Glucose (g/L) | 43 | 43 |
| Maximum Ethanol (g/L) | 32.2 | 36.1 |
| Ethanol Production (g/Lh) | 0.59 | 0.76 |
| Y_{EtOH/S} (g/g) | 0.32 | 0.36 |

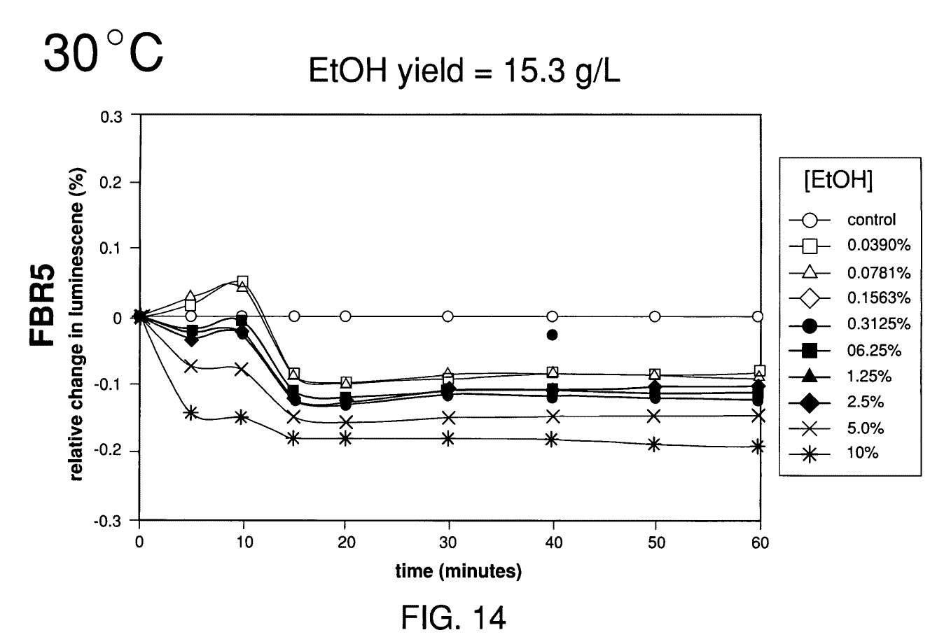

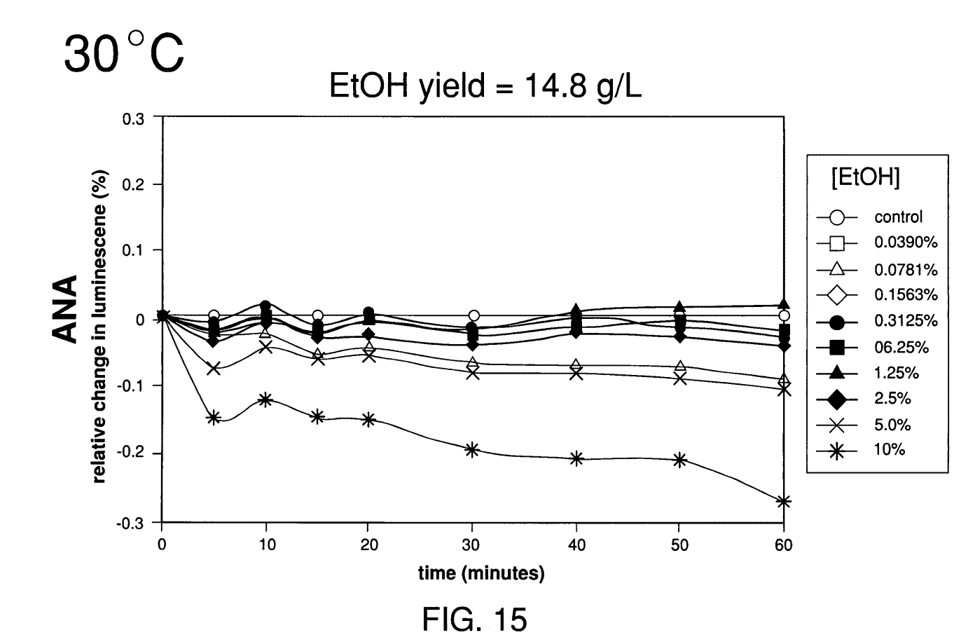

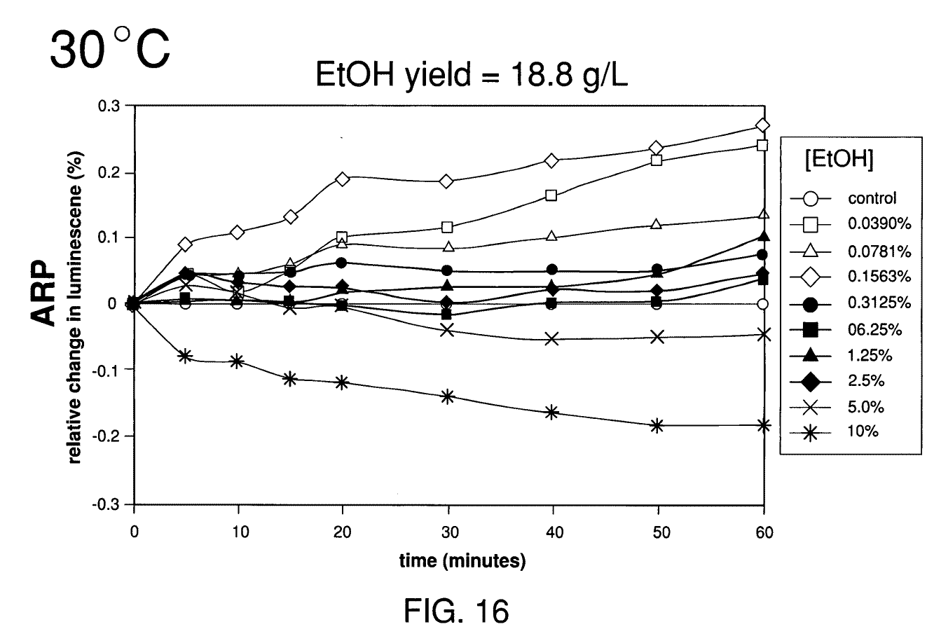

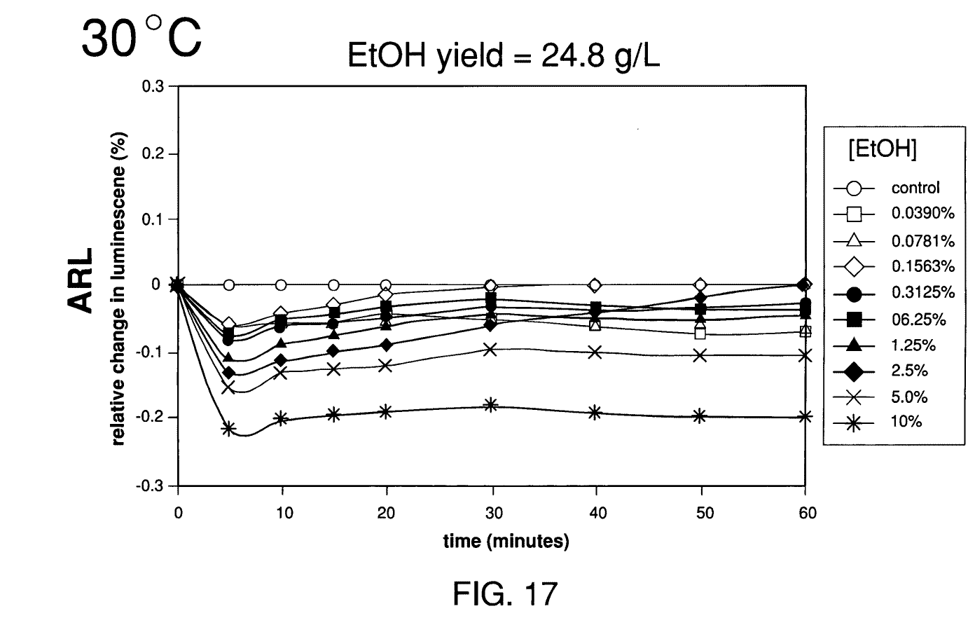

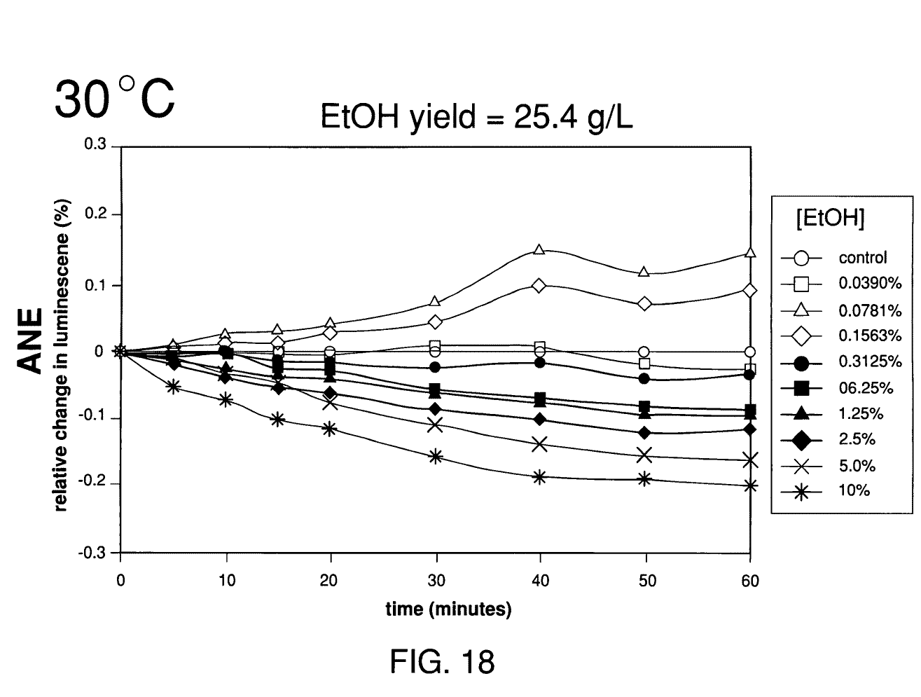

As can be seen in FIG. 17 versus FIG. 14, the FBR5 strain ARL derived according to the present invention realizes an ethanol yield of 24.8 g/L versus 15.3 g/L for FBR5 under comparable fermentation conditions at 30° C., i.e., a 62% increase in ethanol yield. Likewise, the FBR5 strain ANE derived according to the present invention realizes an ethanol yield of 25.4 g/L versus 15.3 g/L for FBR5, i.e., a 66% increase in ethanol yield. The fermentation conditions for FIGS. 14-18 included LB broth with 120 g/L xylose and 100 mg/L ampicillin.

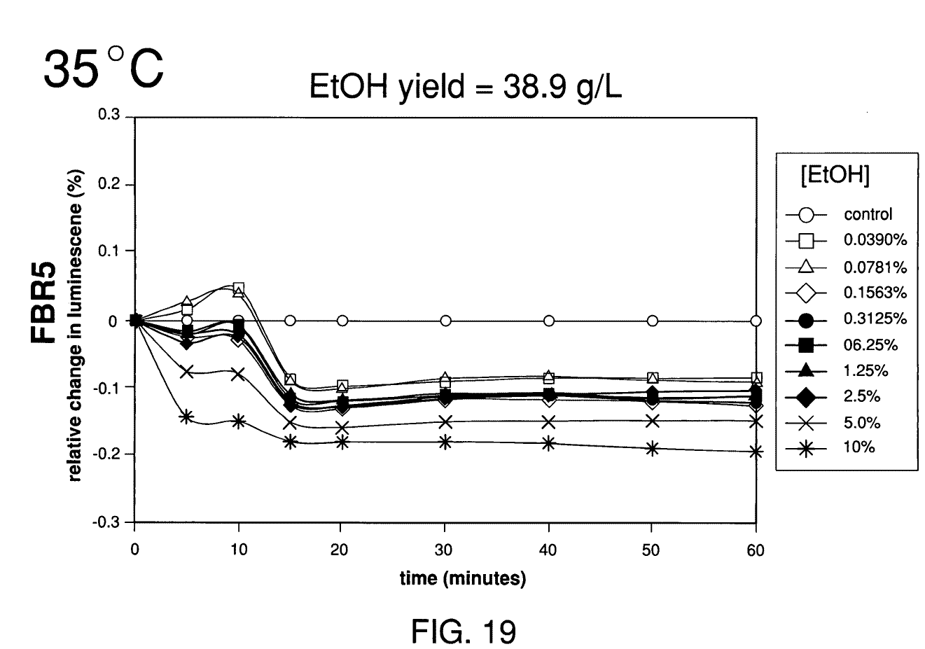

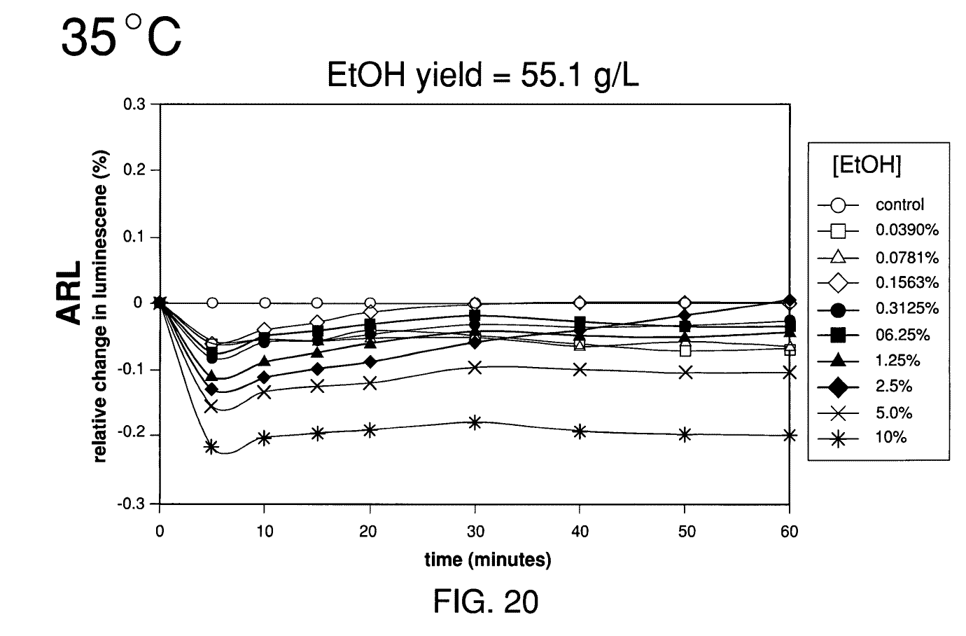

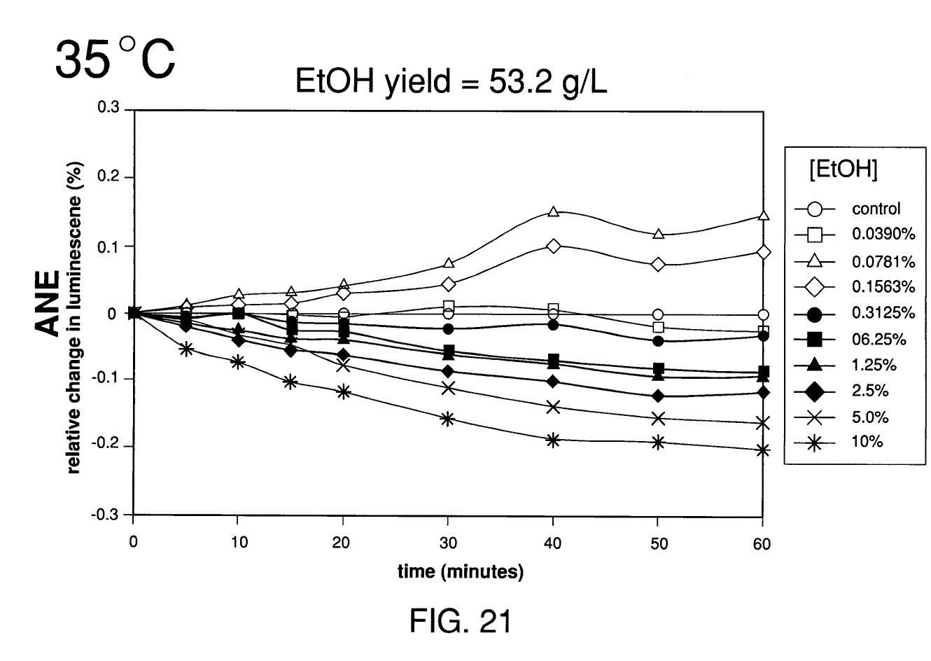

FIGS. 19-21 further support these results. That is, at similar fermentation conditions (except that the fermentation temperature was raised to 35° C.), it is seen that the FBR5 strain ARL derived according to the present invention realized an ethanol yield of 55.1 g/L versus 38.9 g/L for FBR5, i.e., a 41.6% increase in ethanol yield. Likewise, the FBR5 strain ANE derived according to the present invention realized an ethanol yield of 53.2 g/L versus 38.9 g/L for FBR5, i.e., a 36.8% increase in ethanol yield. Perhaps what may be most significant about the results of FIGS. 20 an 21 is that the FBR5 strains ARL and ANE according to the present invention appear to far out-perform their parent FBR5 under similar fermentation conditions notwithstanding their fermentation temperature.

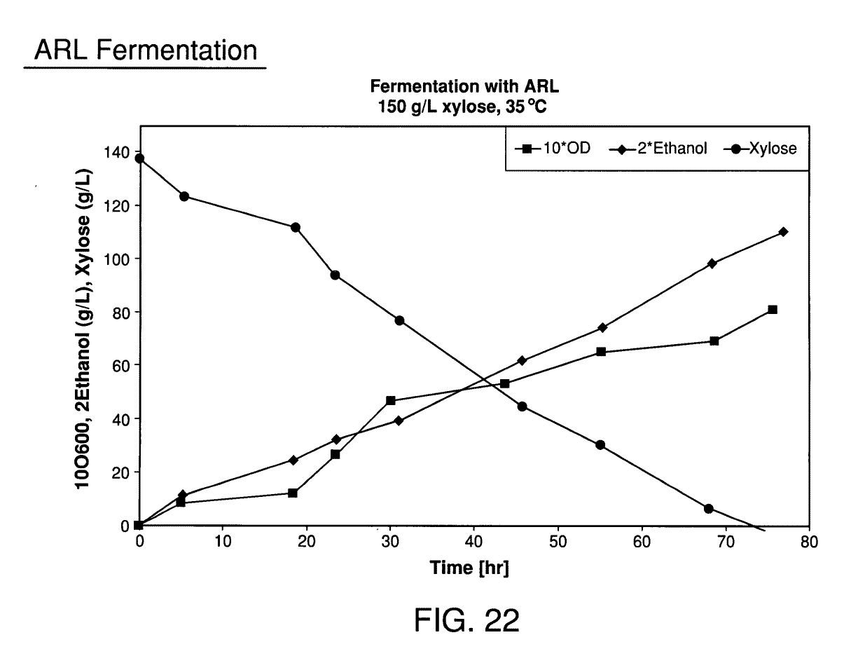

FIG. 22 is a graph of the optical density, ethanol production and xylose concentration under fermentation with E. coli ARL over time in the presence of 150 g/L xylose at 35° C. As can be seen from that figure, ARL is particularly efficient at producing ethanol in the presence of high concentrations of xylose and high fermentation temperatures.

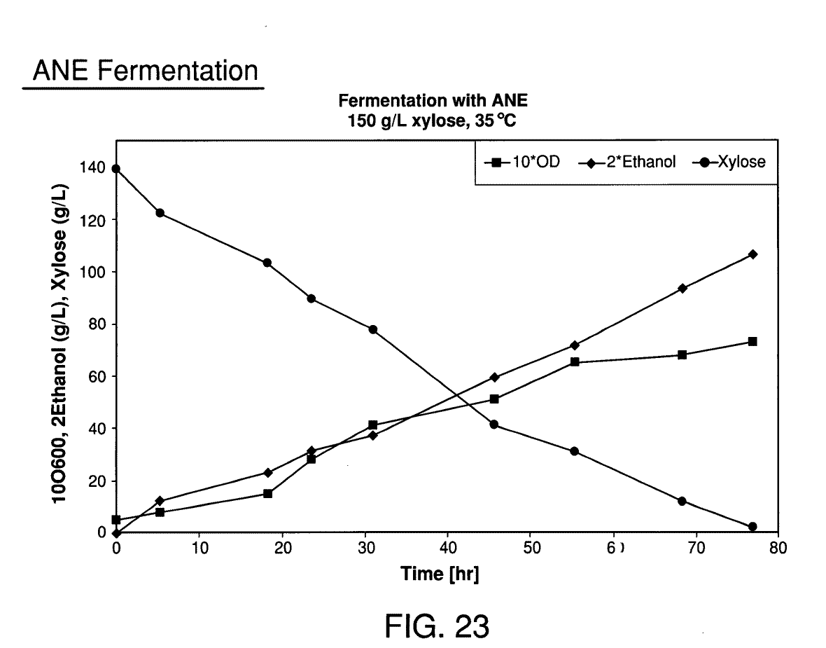

FIG. 23 is a graph of the optical density, ethanol production and xylose concentration under fermentation with E. coli ANE over time in the presence of 150 g/L xylose at 35° C. Similar to FIG. 22, it can be seen that ANE is efficient at producing ethanol in the presence of high concentrations of xylose and high fermentation temperatures.

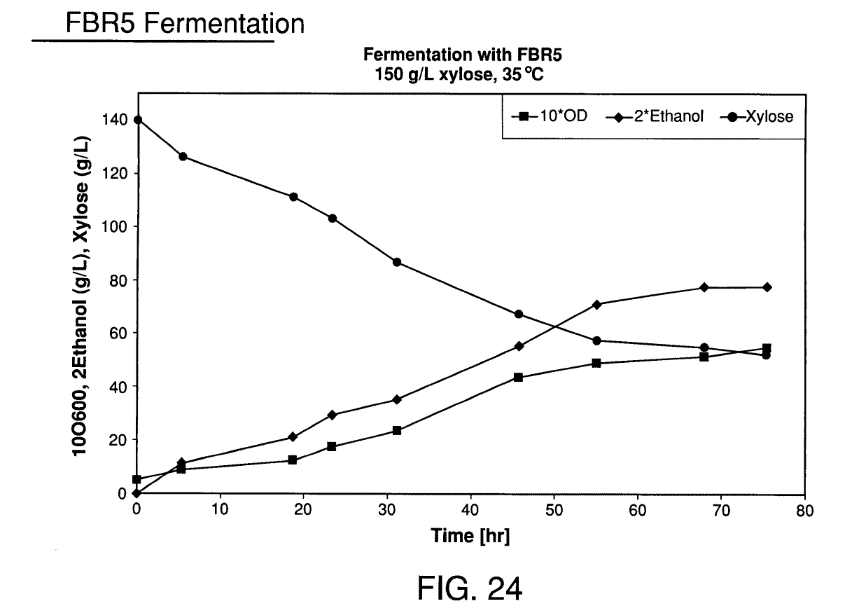

FIG. 24 is a graph of the optical density, ethanol production and xylose concentration under fermentation with E. coli FBR5 over time in the presence of 150 g/L xylose at 35° C. In comparison to FIGS. 22 and 23, FIG. 24 demonstrates that FBR5 is substantially less efficient at producing ethanol in the presence of high concentrations of xylose and high fermentation temperatures than ARL or ANE.

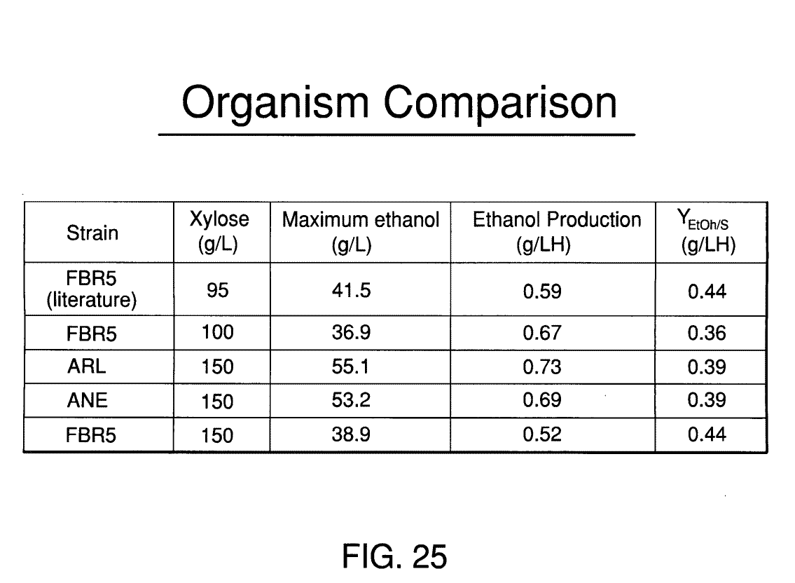

FIG. 25 summarizes the ethanol performance characteristics of ideal strains and actual strains E. coli FBR5 versus the ethanol challenged derivatives thereof produced pursuant to the present invention. As FIG. 25. shows the FBR5 derivatives according to the invention clearly produce more ethanol than their parent E. coli FBR5.

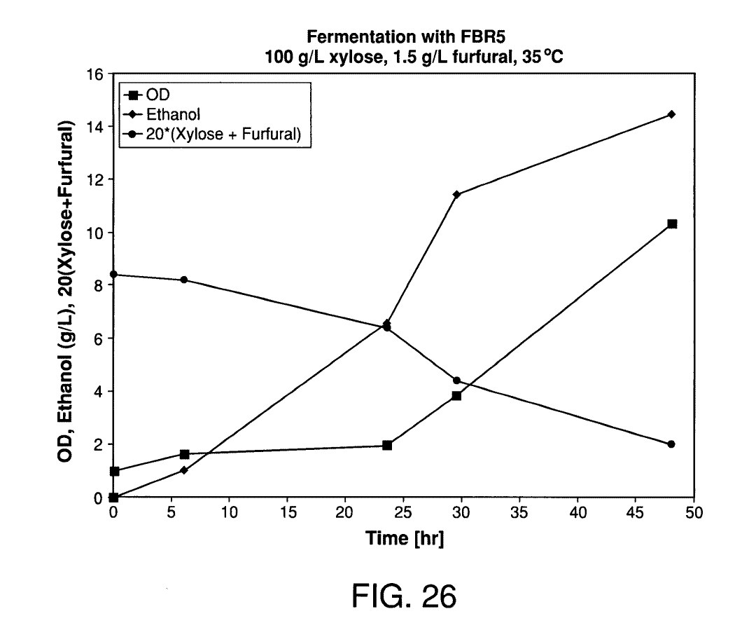

FIG. 26 is a graph of the optical density and ethanol production under fermentation with E. coli FBR5 over time in the presence of 100 g/L xylose and 1.5 g/L furfural at 35° C. FIG. 26 reveals that FBR5 produces approximately 12 g/L ethanol at 30 hours and little over 14 g/L at over 45 hours.

Continuing, another derivative of E. coli FBR5 according to the invention has been shown to exhibit dual resistance to ethanol and furfural. That derivative is identified herein as PS6 (“PS” meaning a “plate selection” furfural resistant strain that is isolated via a furfural challenge in a broth culture).

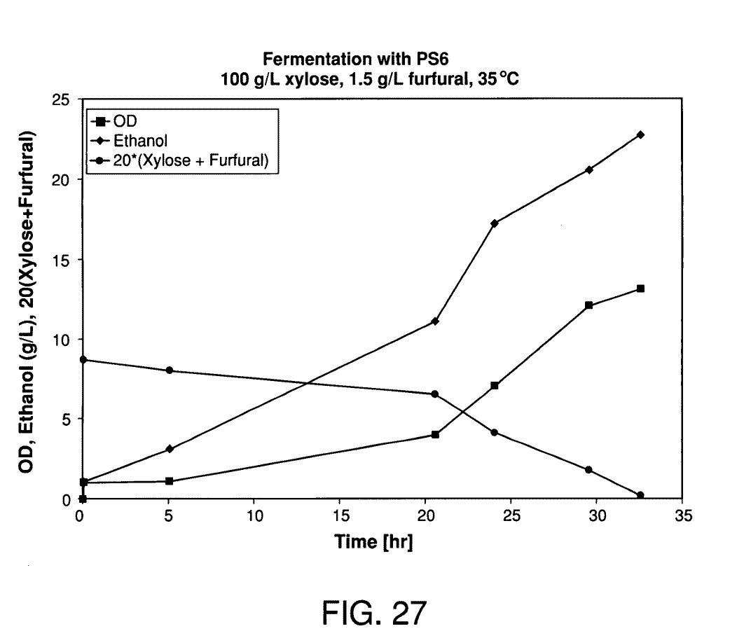

FIG. 27 is a graph of the optical density and ethanol production under fermentation with E. coli PS6 over time in the presence of 100 g/L xylose and 1.5 g/L furfural at 35° C. FIG. 27 reveals that PS6 produces approximately 23 g/L ethanol at 33 hours.

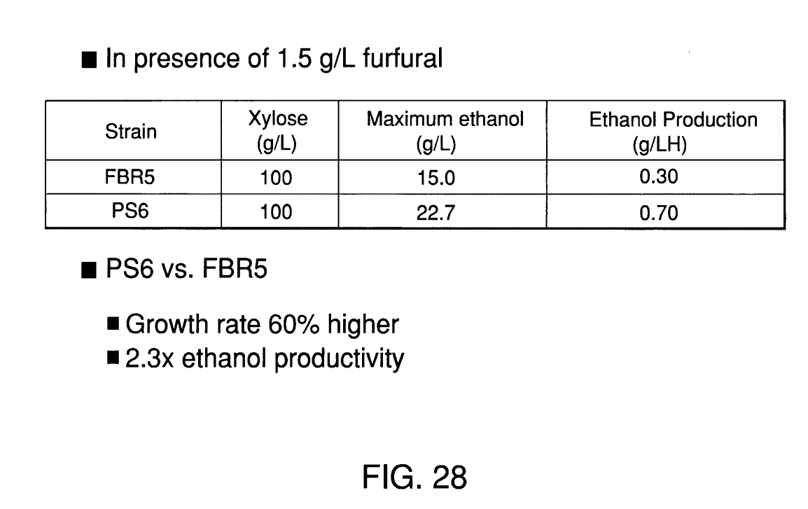

FIG. 28 summarizes the ethanol performance characteristics of E. coli FBR5 and E. coli PS6 in the presence of 100 g/L xylose and 1.5 g/L furfural at 35° C. and clearly shows the advantage of PS6 versus FBR5.

The following materials and methods and methods were used to create and test the PS6 strain. LB containing 50 g/L xylose and 0.1 mg/L of ampicillin was used for all 24 hour growth experiments. Fisher Scientific Lennox LB with 100 g/L xylose and 100 mg/L ampicillin was used for all fermentation experiments. 24 hour growth optical density readings were taken using a Beckmen Coulter DU530 Life Science UV/Vis Spectrophotometer. During fermentations, an HP 8453 spectrophotometer was used to read the optical density. Ethanol readings were determined using a YSI 2700 Select biochemistry analyzer. Xylose readings were determined by a revised version of a method by Eberts. Fermentations were conducted in three fermentors: a 2.5L Wheaton MBF, a 3L New Brunswick Scientific (NBS) BioFlo 3000, and a 2L NBS BioFlo C-30. Three simultaneous fermentations of FBR5 conducted in each fermentor indicated that the variation in fermentors was negligible and therefore runs completed in different fermentors are directly comparable.

Overnight cultures were first grown for 24 hours. These cultures were then transferred into 250 mL flasks each containing 50 mL of LB media. Each flask was grown to an optical density of 2.0 at a wave length of 550 nm. Eleven challenge media bottles of sterile LB were prepared with each bottle containing an increasing concentration of ethanol from 0 to 65 g/L. Eleven 100×13 mm test tubes were filled with 4 mL of the eleven concentrations of challenge media. Once an optical density of 2.0 was reached 50 μL aliquots were pipetted into each test tube. Samples were incubated at 30° C. for 24 hours and the optical density was read at a wavelength of 550 nm. These relative growth values were normalized uninhibited growth.

For each fermentation 1 L of Lennox LB was prepared and autoclaved in the respective vessel. A 300 mL solution of 130 g xylose and 6 g Lennox LB was sterile filtered. An ampicillin stock solution of 0.13 g/mL was added to each fermentor to maintain an overall ampicillin concentration of 100 mg/mL. Additionally, 1.68 mL of furfural was added to each fermentor to achieve a 1.5 g/L concentration of furfural. All fermentations were conducted at 35° C. with agitation set at 300 RPM. The pH of each vessel was automatically maintained at 6.5 by the addition of 2.0N KOH. The temperatures and pH were monitored throughout each fermentation. The dissolved oxygen concentration (DO) was monitored in one vessel. Air was sparged through each fermentor before inoculation. The air supply was terminated once each vessel was inoculated simultaneously. Once the monitored DO of the monitored vessel equilibrated at 0%, a nitrogen purge was activated for each vessel. This procedure was implemented to determine if the consumption of oxygen in the headspace of the fermentor varied between the parent strain and the furfural resistant strains.

Samples were taken before and after inoculation and 2-3 times a day while the fermentations were being conducted. The OD was read at a wavelength of 600 and 650 nm. Four 1 mL samples were centrifuged at each sampling point. The supernatant from these samples was frozen for later analysis. The ethanol concentration was also determined. The aldehyde concentration was determined using a xylose assay.

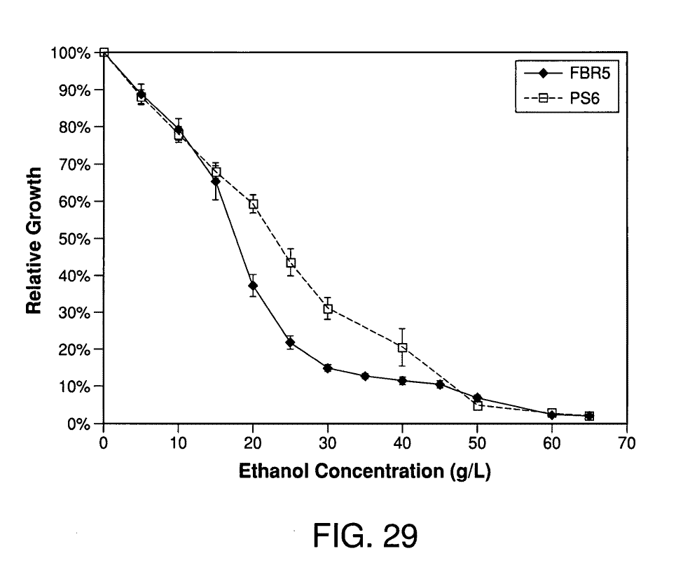

FIG. 29 is a graph of the relative growth of E. coli PS6 versus E. coli FBR5 as a function of exposure to ethanol. From ethanol concentrations of from about 15% to about 50% PS6 exhibits substantially greater cell growth than FBR5. The results shown in FIG. 29 were conducted in replicates of 12 or more for each strain. From 20 to 40 g/L ethanol PS6 demonstrates statistically substantially greater growth after 24 hours. Furfural resistance is known to be a furfural breakdown pathway from furfural to furfural alcohol or furoic acid which detoxifies furfural. This phenotypical difference does not account for the demonstrated ethanol resistance. Without being bound to theory, one possible explanation for this cross resistance is an increase in the protein to lipid ratio in the outer membrane. This would restrict the permeation of furfural into the cell to allow for less glycolosis inhibition during furfural breakdown which would result in furfural resistance. Likewise, ethanol is believed to interfere with hydrophobic forces between the phospholipids in the membrane causing cellular leakage. This proposed reduction in phospholipids would reduce the effect ethanol has on the membrane and account for the demonstrated cross resistance to exogenous ethanol.

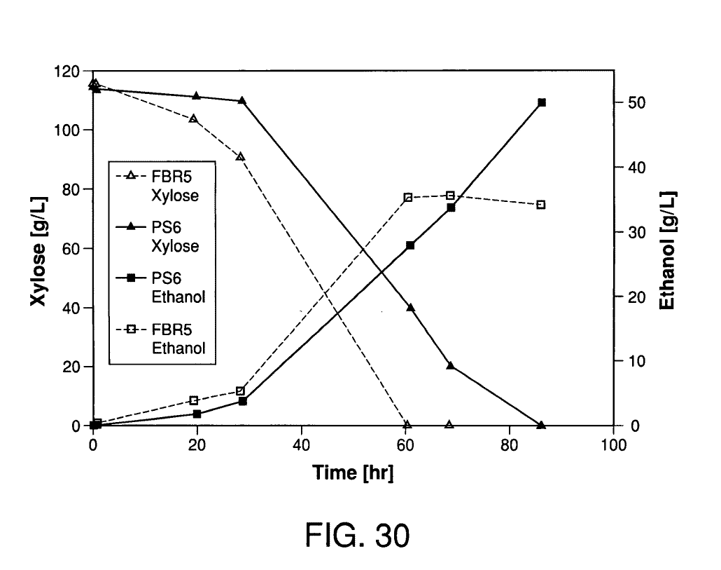

Whether furfural resistance can produce a cross resistance to ethanol fermentation data is an issue. Xylose consumption and ethanol production are the chief concerns in fermentation performance. FIG. 30 is a graph of ethanol production and xylose concentration under fermentation of E. coli PS6 versus E. coli FBR5 over time in the presence of approximately 100 g/L xylose and 1.5 g/L furfural. That figure reveals that although PS6 takes longer to consume xylose than FBR5, it produces a far greater yield of ethanol than FBR5.

These fermentation results show FBR5 that consumes the available xylose before the PS6 strain, yet FBR5 produces 15 g/L less ethanol than PS6. The difference in final ethanol concentration supports the same hypothesis as the bench top 24 hour growth results. The maximum ethanol concentration FBR5 reached was 35 g/L which was within the deviation range shown in the 24 hour growth results. Also notable is the difference in yield defined in Equation (2), supra.

Even though FBR5 consumed the xylose more rapidly it did so only with a yield of 0.29 g ethanol/g xylose. PS6 produced a higher yield of 0.42 g ethanol/g xylose which is believed to be attributable to its cross resistance to ethanol.

Likewise, in addition to demonstrating the anticipated furfural resistance, PS6 has demonstrated a cross resistance to ethanol. This has been demonstrated in both bench top and fermentation exercises. A possible explanation for this cross resistance may lie in the membrane composition of the PS6 mutant. However, it is clear that PS6 can survive more readily in ethanol and produce more ethanol from xylose than the parent strain FBR5.

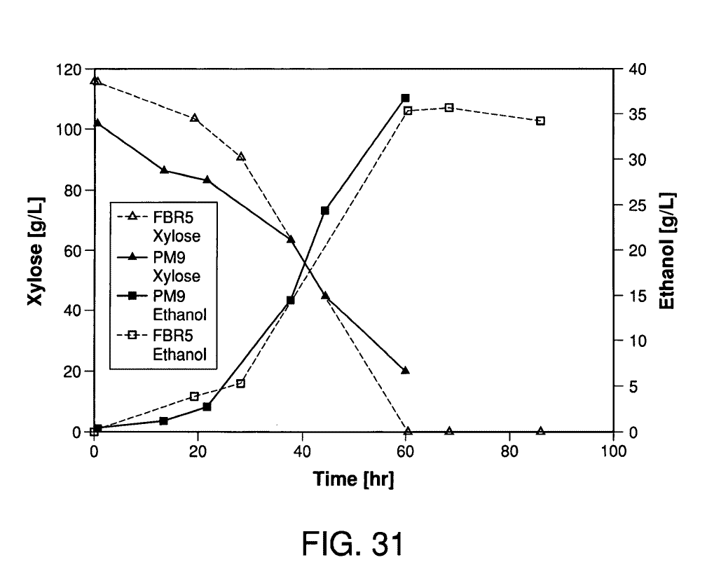

FIG. 31 illustrates the performance characteristics of another challenged E. coli FBR5 strain developed according to the present invention. That strain, identified herein as “PM9”, is an ethanologically and furfuralogically challenged E. coli strain. “PM” stands for “PS6 Mutant”. That is to say, PM9 is a ethanol challenged strain of PS6 which itself is an furfural challenged strain of FBR5. As revealed in FIG. 31, PM9 also outperforms FBR5 in terms of ethanol production.

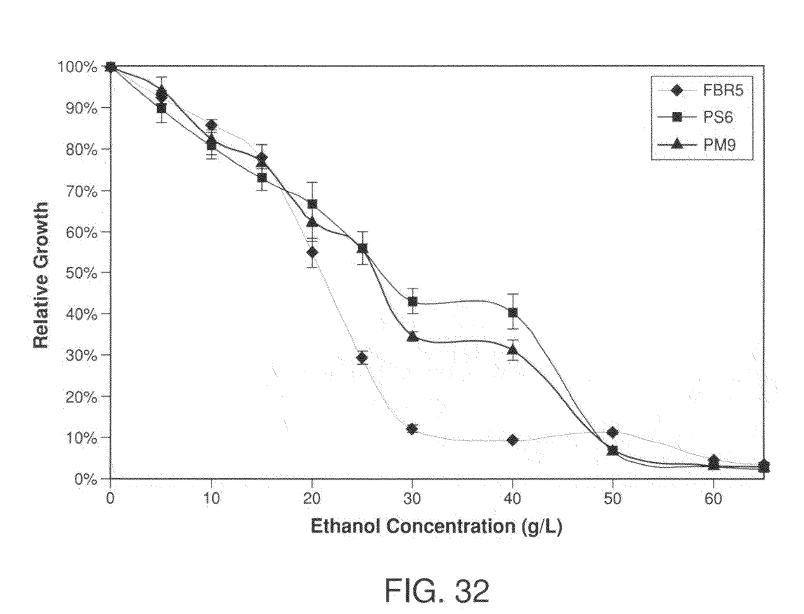

Turning to FIG. 32, there is shown a graph of the relative growth of E. coli PS6 and PM9 versus E. coli FBR5 as a function of exposure to ethanol. Similar to FIG. 29, from ethanol concentrations of from about 15% to about 50% it is seen that PM9, like PS6, exhibits substantially greater cell growth than FBR5.

Although the invention has been described in detail for the purpose of illustration, it is to be understood that such detail is solely for that purpose and that variations can be made therein by those skilled in the art without departing from the spirit and scope of the invention as claimed herein.

What is claimed is:

1. An ethanol and furfural challenged strain of Escherichia coli (E. coli) FBR5 capable of producing a greater quantity of ethanol from fermentation of cellulosic biomass than E. coli FBR5.

2. The strain of claim 1 wherein the strain is aerobic.

3. The strain of claim 1 wherein the strain is anaerobic.

4. The strain of claim 1 wherein the cellulosic biomass is corn stover.
